(19) United States
(12) Patent Application Publication
(10) Pub. No.: US 2016/0213504 A1
(43) Pub. Date: Jul. 28, 2016

(54) SLEEVE WITH EXTERNAL ASSISTANCE DURING AN ARM MOTION

(71) Applicant: Jason Colleran, Chamblee, GA (US)

(72) Inventor: Jason Colleran, Chamblee, GA (US)

(21) Appl. No.: 15/089,564

(22) Filed: Apr. 3, 2016

Related U.S. Application Data
(63) Continuation-in-part of application No. 14/541,259, filed on Nov. 14, 2014.

(51) Int. Cl. A61F 5/01

(52) U.S. Cl. CPC A61F 5/013

(57) ABSTRACT

Systems and methods for providing external assistance during a throwing motion include a detachable sleeve comprising a torso and an elastomer mesh arm region. The arm region comprises a long sleeve that covers the user's throwing arm to the middle of the forearm. The arm region may include an opening in the elbow portion hereof large enough for the elbow of a user to extend through. In this way, the elastomer mesh of the arm region will not interfere with the rotation of the elbow during a throwing motion. During a throwing motion, the elastomer mesh of the arm region helps prevent an overextension of the UCL and assists the varying muscles in the arm and shoulder that provide stability to the UCL during the forward motion of the arm leading to the release of the ball (or other object thrown).

### CROSS REFERENCE TO RELATED APPLICATION

This is a continuation-in-part application claiming the benefit of U.S. patent application Ser. No. 14/541,259, which was filed on Nov. 14, 2014, and is incorporated herein by reference in its entirety.

### TECHNICAL FIELD

This disclosure relates to implementations of a sleeve with external assistance during an arm motion.

### BACKGROUND

Arm motions, such as a throwing motion when pitching a baseball, may involve a winding-up motion, an acceleration of the arm, release/hitting of a ball, and a follow-through. When the arm is accelerating and following through, a large valgus load may be placed on the elbow joint, and in particular on the medial structures of the elbow including the medial collateral ligament, otherwise known as the ulnar collateral ligament (UCL).

Placing large valgus loads, such as through repeated hard throwing or pitching on the elbow can lead to injury to the UCL, including over-stretching, fraying, and tearing. The UCL may become injured either by acute ligament tears, or merely through overuse and repetitive stress. For example, the repeated valgus stresses from repetitive throwing may result in overuse injury where the tissue breakdown exceeds the tissues ability to repair itself. In children such repetitive stress may manifest as “little league elbow” which can lead to medial epicondylar apophysitis and stress fractures through the medial epicondylar epiphyses.

Injury to the UCL can be particularly problematic because the UCL along with another ligament is the main source of stability for the elbow. When the UCL is damaged or injured and does not heal correctly the elbow can become loose and/or unstable leading to further injury. This may lead to the need for corrective surgery, such as Tommy John surgery to repair the UCL.

Previous attempts at preventative or rehabilitation devices or garments have focused on either compression, such as by a tight fitting sleeve over the elbow, or braces designed to provide support and/ or limit the arm motion. However, such prior approaches are focused on trying to provide structural support for the elbow, rather than providing positive assistance to the muscles in the arm and shoulder that help stabilize the UCL in order to reduce the valgus load.

Therefore, it would be desirable to provide a system and method for external assistance to the muscles in the arm and shoulder that stabilize the UCL during the throwing motion for prevention of and/or rehabilitation from UCL injuries.

### SUMMARY

Embodiments of systems and methods for providing external assistance for an arm motion, such as a throwing motion are provided. An exemplary system includes a detachable sleeve comprising a torso region configured to receive at least part of a torso of a user and an arm region configured to receive at least part of an arm of the user. The exemplary system also includes a first elastic portion with a first end and a second end. The first end of the first elastic portion is affixed to a front side of the torso region and the second end of the first elastic portion is affixed to a top surface of the arm region. The exemplary system further includes a second elastic portion with a first end and a second end. The first end of the first elastic portion is affixed to a back side of the torso region and the second end of the first elastic portion is affixed to the top surface of the arm region. The exemplary system finally includes a third elastic portion with a first end, a second area, a third area, and a second end. The first end of the third elastic portion is affixed to the torso region, the second area is disposed on the top surface of the arm region, the third area is disposed on a bottom surface of the arm region, and the second end of the third elastic portion is configured to encircle a throwing hand of the user.

Other embodiments are also provided. Other systems, methods, features, and advantages of the invention will be or become apparent to one with skill in the art upon examination of the following figures and detailed description. It is intended that all such additional systems, methods, features, and advantages be included within this description, be within the scope of the invention, and be protected by the accompanying claims.

### BRIEF DESCRIPTION OF THE FIGURES

Exemplary embodiments of the invention can be better understood with reference to the following figures. The components within the figures are not necessarily to scale, emphasis instead being placed upon clearly illustrating the principles of the invention. Moreover, in the figures, like reference numerals designate corresponding parts throughout the different views.

FIG. 1 illustrates a front view of an exemplary embodiment of a system that provides external assistance for an arm motion.

FIG. 2 illustrates a rear view of aspects of the exemplary embodiment of the system shown in FIG. 1.

FIG. 3 illustrates a front view of another exemplary embodiment of a system that provides external assistance for an arm motion.

FIG. 4 illustrates a rear view of aspects of the exemplary embodiment of the system shown in FIG. 3.

FIG. 5 illustrates a front view of yet another exemplary embodiment of a system that provides external assistance for an arm motion.

FIG. 6 illustrates a rear view of aspects of the exemplary embodiment of the system shown in FIG. 5.

### DETAILED DESCRIPTION

As discussed above an arm motion, such as a throwing motion when pitching a baseball, generally involves a winding-up motion, an acceleration of the arm, release of the ball, and a follow-through. Other similar arm motions may include a swinging motion, such as tennis, handball, volleyball, etc. that generally involve a winding-up motion, an acceleration of the arm, hitting the ball, and a follow-through. When the arm is accelerating and following through, large valgus loads may be placed on the elbow joint, and in particular on the medial structures of the elbow including the medial collateral ligament, otherwise known as the ulnar collateral ligament (UCL).

The below description and accompanying figures disclose and discuss various illustrative embodiments of a sleeve that that provides external assistance for the arm and shoulder to help stabilize the UCL during valgus loads in an arm motion, such as a swinging or throwing motion. Although discussed in the context of a throwing motion, such as pitching a baseball, the below discussion is equally applicable to other similar arm motions, including the swinging motion such as in tennis, handball, volleyball, etc. The disclosed system and methods assist in the prevention of and/or the rehabilitation from UCL injuries, including acute ligament tears/injuries and injuries caused by overuse and/or repetitive stress. As disclosed below in various embodiments, the system and methods may be implemented by a stand-alone device or may be integrated into a garment such as a compression shirt for example.

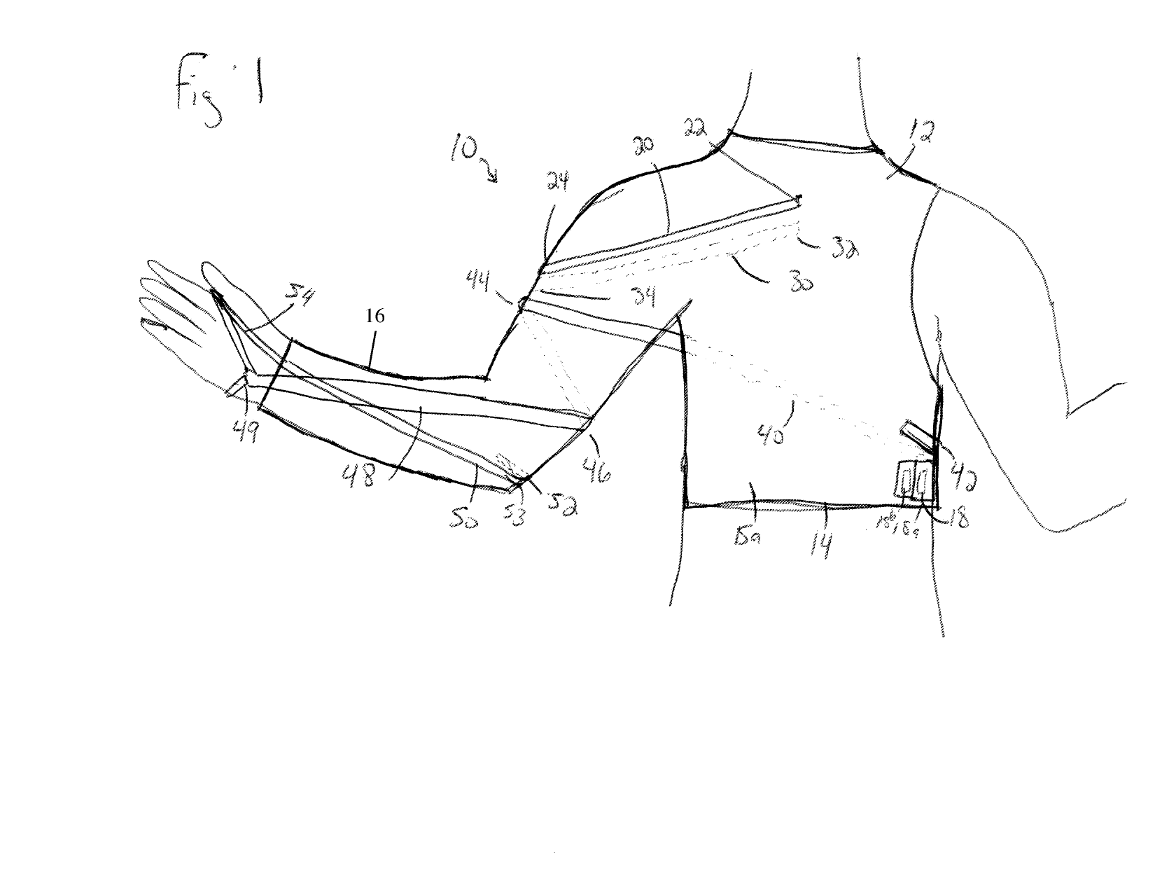

A front view of an exemplary embodiment of a system 10 that provides external assistance for a throwing motion is illustrated in FIG. 1. As shown in FIG. 1, the system 10 includes a detachable sleeve 12 configured to be worn on the throwing arm of a user. In the embodiment illustrated in FIG. 1, the detachable sleeve 12 is configured and adapted to be worn on the right arm; however in the event that the user throws left handed, the detachable sleeve 12 may be configured and adapted to be worn on the left arm if desired as would be understood by one of ordinary skill in the art.

The detachable sleeve 12 shown in FIG. 1 includes a torso region 14 configured to be attached to the torso of the user and an arm region 16 configured to cover at least a portion of the throwing arm of the user when the detachable sleeve 12 is in use. In the embodiment illustrated in FIG. 1, the arm region 16 comprises a long sleeve that covers the user's throwing arm to the wrist. However, in other implementations, the arm region 16 may be shorter and may not extend to the user's wrist.

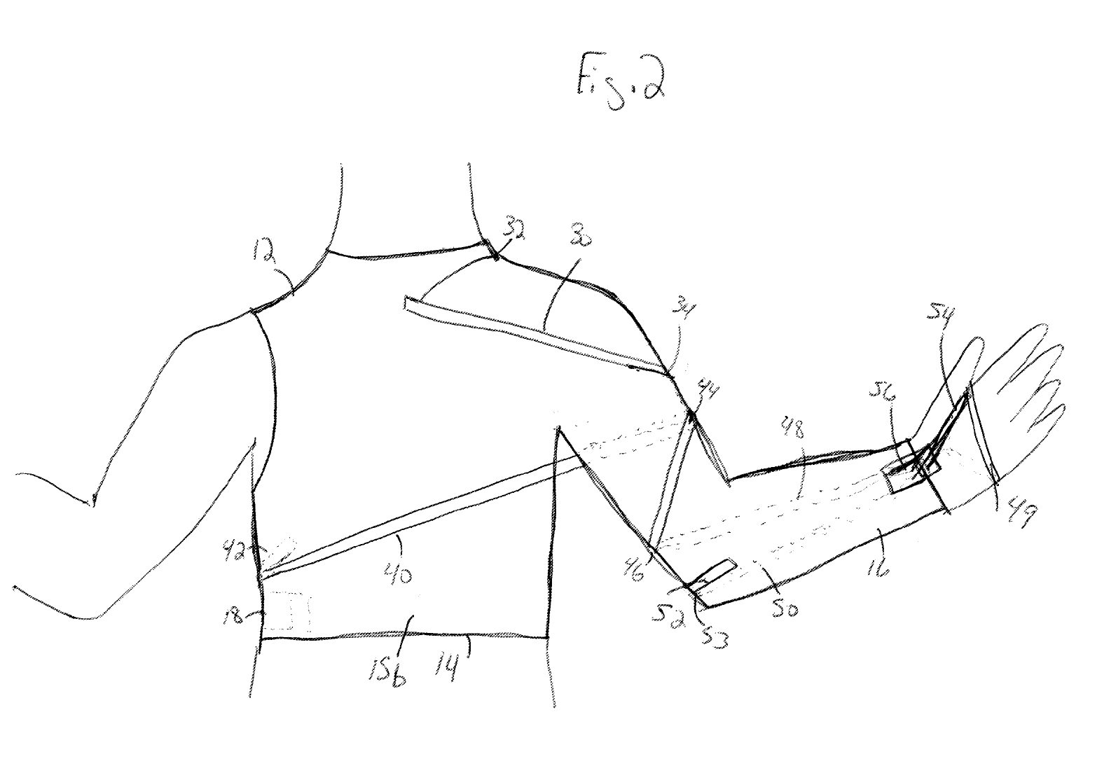

The torso region 14 includes a front side 15a and a back side 15b (see FIG. 2) that can be removably attached together by a connector 18 when the detachable sleeve 12 is worn by a user. The connector 18 may be any appropriate mechanism or method of securely attaching the front side 15a to the back side 15b when user places his or her arm into the detachable sleeve 12. Examples of the connector 18 include snaps, hooks, hook-and-loop fasteners, buttons, etc. capable of adjustably securing the front side 15a to the back side 15b. In the illustrated embodiment of FIG. 1, the connector 18 is located on the front side 15a such that a portion of the back side 15b of the torso region 14 is wrapped around the torso of a user and attached to the front side 15a of the torso region 14, at the connector 18, securing the detachable sleeve 12 to the user. In other embodiments, the connector 18 may be located at the back side 15b, on the side of the user's torso, or anywhere that allows for the detachable sleeve 12 to be secured to the user. Additionally, the connector 18 may comprise multiple connectors 18a and 18b that allow the detachable sleeve 12 to be adjustably secured to fit on torso of varying sizes, lengths, etc. and/or or to allow the other portions of the system 10 described below to be properly positioned on a user.

The detachable sleeve 12 may be made of any appropriate material, such as fabric, neoprene, mesh, or any other desired material that will allow for a snug fit to a user when the detachable sleeve 12 is worn. Accordingly, the material of the detachable sleeve 12 may be relatively elastic as desired to allow for the snug fit. Additionally, in some implementations the torso region 14 of the detachable sleeve 12 may comprise a different material, different thickness of material, different density of material, different elasticity, etc. than the arm region 16 of the detachable sleeve 12.

Thus, by way of a non-limiting example, in one implementation the arm region 16 and the torso region 14 may comprise a single piece of a material such as neoprene. While in a different implementation, the arm region 16 may comprise a first material, a first density of material and/or a first thickness of material, while the torso region 14 may comprise a second material, a second density of material and/or a second thickness of material as desired. Additionally, one or more of the arm region 16 and torso region 14 may include inserts, cut-outs, slits, etc. to allow for moisture and/or air ventilation as desired.

In the embodiment illustrated in FIG. 1, the system 10 includes a first elastic portion 20 with a first end 22 that is affixed to the front side 15a of the torso region 14. The first elastic portion 20 extends to the arm region 16 at the front of the bicep of the user's throwing arm and ends at a second end 24 that is affixed to the arm region 16 at a location that corresponds to the bicep region of the user's throwing arm. In an embodiment, the second end 24 ends and is affixed at a location approximately halfway down the user's lateral humerus bone and/or deltoid tuberosity. The first elastic portion 20 can have any desired width, thickness and/or elasticity. For example, the width, thickness, and/or elasticity of the first elastic portion 20 may vary depending on the size, age, gender, extent of injury, etc. of a user.

In the embodiment shown in FIG. 1, the first elastic portion 20 comprises a strap or band external to the detachable sleeve 12 with the first end 22 and second end 24 permanently affixed to the exterior of the detachable sleeve 12. The rest of the first elastic portion 20 may not be affixed to the detachable sleeve 12 in some implementations of this embodiment to allow the first elastic portion 20 to be positioned as desired to accommodate users with varying arm sizes, lengths, etc. Alternatively, in other embodiments, the first elastic portion 20 may be removably secured to the exterior of the detachable sleeve 12 in order to ensure that the first elastic portion 20 is properly positioned. For example, a hook-and-loop enclosure or other attaching mechanism may be used at the second end 24 in order to ensure that the first elastic portion 20 is properly positioned when wearing and/or using the detachable sleeve 12.

In yet other embodiments, more than the first end 22 and the second end 24 of the first elastic portion 20 may be affixed to the detachable sleeve 12. For example, in some implementations, the first elastic portion 20 may be contained in a channel or other guiding portion of the detachable sleeve 12 to secure the first elastic portion 20 in position. While in other implementations, the detachable sleeve 12 may comprise multiple layers of material with the first elastic portion 20 located between the layers of the detachable sleeve 12 in order to secure the first elastic portion 20 into position. In such embodiments, the first elastic portion 20 may be properly positioned on the user by adjustment of the fit of the detachable sleeve 12 on the user. Additionally, as would be understood, the detachable sleeve 12 may be provided in varying sizes and/or configurations to accommodate the size, age, and/or gender of the user.

The system 10 also includes a second elastic portion 30 with a first end 32 affixed to the back portion 15b of the torso region 14. In a preferred embodiment, the first end 32 is affixed to the back portion 15b of the torso region 14 at a location corresponding generally to a scapula of the user, and more particularly in some implementations at a location slightly inferior and medial of the superior angle of the scapula between the medial border of the scapula and the spinous processes of the thoracic spine. The second elastic portion 30 (see FIG. 2) extends to the arm region 16 at the bicep of the user's throwing arm and ends at a second end 34 that is affixed to the arm region 16 at a location that corresponds to the bicep region of the user's throwing arm. In an embodiment, the second end 34 ends and is affixed at a location approximately halfway down the user's lateral humerus bone and/or deltoid tuberosity.

As with the first elastic portion 20, the second elastic portion 30 may have any desired width, thickness and/or elasticity. For example, the width, thickness, and/or elasticity of the second elastic portion 30 may vary depending on the size, age, gender, extent of injury, etc. of a user. Additionally, the second elastic portion 30 may contain additional mechanisms or may be configured to removably or permanently affix parts of or the entire second elastic portion 30 into place on the detachable sleeve 12 when worn and/or used by the user. For example, a hook-and-loop fasteners or other attaching mechanism may be used at the second end 34 in order to ensure that the second elastic portion 30 is properly positioned when wearing and/or using the detachable sleeve 12.

Additionally, or alternatively, the second elastic portion 30 may be contained in a channel or other guiding portion of the detachable sleeve 12 to secure the second elastic portion 30 in position. In some implementations the detachable sleeve 12 may comprise multiple layers of material with the second elastic portion 30 located between the layers of the detachable sleeve 12 in order to secure the second elastic portion 30 into position. In such embodiments, the second elastic portion 30 may be properly positioned on the user by adjustment of the fit of the arm region 16 and/or the detachable sleeve 12 on the user. Additionally, as would be understood, the arm region 16 and/or detachable sleeve 12 may be provided in varying sizes and/or configurations to accommodate the size, age, and/or gender of the user.

The illustrated system 10 of FIG. 1 also includes a third elastic portion 40 with a first end 42 adjustably and/or removably affixed to the torso region 14 of the detachable sleeve 12. The first end 42 may be adjustably affixed to the torso region 14 such as by use of a hook-and-loop mechanism or other desired mechanism so that the first end 42 may be adjusted in a manner to allow the tension in the third elastic portion 40 to be lessened or increased as desired.

In the embodiment illustrated in FIG. 1, the first end 42 is illustrated as removably attaching to the front portion 15a of the torso region 14. However, this is for illustrative purposes only and the disclosure is not limited to the location of the first end 42 illustrated in the figures. In other embodiments, the first end 42 may removably or adjustably attach to the back portion 15b (see FIG. 2) of the torso region 14. Additionally, the first end 42 may be located anywhere on the detachable sleeve 12 that will provide the desired benefits, including those discussed below. Thus, the position of the first end 42 may vary depending on multiple factors including the amount of tension desired in the third elastic portion 40 during the throwing motion, the skill of the user, the age, size, gender, etc. of the user, whether the system 10 is being used to prevent injury or as part of a rehabilitation program, etc. All such additional embodiments are within the scope of this disclosure.

The illustrated third elastic portion 40 extends from the first end 42 to the back portion 15b of the torso region 14 on the user's non-throwing arm side. The third elastic portion 40 continues across the width of the back portion 15b of the torso region, across the front of the arm region 16 to a second area 44 located on the arm region 16 at approximately the bicep area of the user. The third elastic portion 30 continues, wrapping around the bicep area to the back of the arm region 16 to a third area 46 located on the arm region 16 at approximately the tricep area of the user. In an embodiment, the third area 46 is located approximately at the distal humerus.

From the third area 46, the third elastic portion 40 continues with a fourth area 48 toward the palm of the user's throwing hand to a second end 49 of the third elastic portion 40. The second end 49 is past the end of the arm region 16 of the detachable sleeve and generally comprises a loop traversing the width of the palm of the user's throwing hand and passing between the thumb and first finger of the user's throwing hand to encircle the user's throwing hand as illustrated in FIGS. 1 and 2. In the illustrated embodiment, the third elastic portion 40 is a single component beginning at the first end 42 and terminating at the second end 49 that encircles the user's hand (see FIG. 2). In other embodiments, the third elastic portion 40 may comprise one or more separate components having the general configuration illustrated in FIG. 1 (and/or FIG. 2). For example the second end 49 may be a separate elastic or other material loop that is affixed to the end of the fourth area 48 of the third elastic portion 40.

The third elastic portion 40 may have any desired width, thickness and/or elasticity. For example, the width, thickness, and/or elasticity of the third elastic portion which may vary depending on the size, age, gender, extent of injury, etc. of a user. Additionally, while only the first end 42 of the third elastic portion 40 has been discussed as being removably affixed to the detachable sleeve 12, one of ordinary skill in the art would understand that one or more of the second area 44, third area 46 and/or fourth area 48 could be permanently or removably affixed to the detachable sleeve 12. For example, in some embodiments, to assist with the proper positioning of the third elastic portion 40, the second area 44 may be positionable and removably affixable to the proper location of the arm region 16 of the detachable sleeve 12, such as by one or more hook-and-loop fasteners, hooks, buttons, etc. as desired. The same is also true for one or more of the third area 46 and/or the fourth area 48 of the third elastic portion 40. Additionally, or alternatively, one or more of the second area 44, third area 46, and/or fourth area 48 may be permanently affixed to the detachable sleeve 16 such that the user positions these areas 44, 46, 48 by adjusting the fit of the detachable sleeve 12, such as by the connector 18.

Similarly, one or more parts of the third elastic portion 40 may be contained in a channel or other guiding portion of the detachable sleeve 12 to secure the third elastic portion 40 in position. For example, in some implementations the detachable sleeve 12 may comprise multiple layers of material with parts of the third elastic portion 40 located between the layers of the detachable sleeve 12 in order to secure the third elastic portion 40 into position. In such embodiments, the third elastic portion 40 may again be properly positioned on the user by adjustment of the fit of the arm region 16 and/or the detachable sleeve 12 on the user. Additionally, as would be understood, the arm region 16 and/or detachable sleeve 12 may be provided in varying sizes and/or configurations to accommodate the size, age, and/or gender of the user.

The illustrated system 10 of FIG. 1 may also include a fourth elastic portion 50 with a first end 52 affixed to back side of the arm region 16 of the detachable sleeve 12 as illustrated in FIG. 1. The first end 52 is affixed at a position generally located at the lateral shaft of the radius of the user's throwing arm, or about 2-6 inches from the elbow of the user's throwing arm. The fourth elastic portion 50 includes a middle area 53 generally positioned to wrap around the posterior elbow joint of the user's throwing arm and continue up along the ulna of the user's throwing arm as illustrated in FIG. 1. The fourth elastic portion 50 includes a second end 54 configured to extend past the end of the sleeve region 16 of the detachable sleeve 12, between the thumb and first finger of the user's throwing hand to an attachment portion 56 located at the back of the user's throwing hand (see FIG. 2).

The fourth elastic portion 50 may have any desired width, thickness and/or elasticity. For example, the width, thickness, and/or elasticity of the fourth elastic portion may vary depending on the size, age, gender, extent of injury, etc. of a user. Additionally, in some embodiments the first end 52 of the fourth elastic portion 50 may be permanently affixed to the arm region 16 of the detachable sleeve 12. In other embodiments the first end 52 may be removably affixed, such as by a hook-and-loop fastener, so as to allow for more precise positioning of the fourth elastic portion 50, to allow for lessening or increasing the tension in the fourth elastic portion 50 when the system 10 is being used, etc.

Similarly, one of ordinary skill in the art would understand that the middle area 53 could be permanently or removably affixed to the detachable sleeve 12. For example, in some embodiments, to assist with the proper positioning of the fourth elastic portion 50, the middle area 53 may be positionable and removably affixable to the proper location of the arm region 16 of the detachable sleeve, such as by one or more hook-and-loop fasteners, hooks, buttons, etc. as desired. Additionally, or alternatively, the middle area 53 may be permanently affixed to the detachable sleeve 16 such that the user positions this area 53 by adjusting the fit of the detachable sleeve 12, such as by the connector 18.

Additionally, or alternatively, one or more parts of the fourth elastic portion 50 may be contained in channel or other guiding portion of the detachable sleeve 12 to secure the fourth elastic portion 50 in position. For example, in some implementations the detachable sleeve 12 may comprise multiple layers of material with parts of the third elastic portion 40 located between the layers of the detachable sleeve 12 in order to secure the fourth elastic portion 50 into position. In such embodiments, the fourth elastic portion 50 may again be properly positioned on the user by adjustment of the fit of the arm region 16 and/or the detachable sleeve 12 on the user. Additionally, as would be understood, the arm region 16 and/or detachable sleeve 12 may be provided in varying sizes and/or configurations to accommodate the size, age, and/or gender of the user.

Note that in varying embodiments, one or more of the first elastic portion 20, second elastic portion 30, third elastic portion 40, and/or fourth elastic portion 50 may be made of the same or different materials, may be the same or different thickness or width, may have the same or different amounts (or moduli) of elasticity, etc. as desired and in accordance with the systems and methods disclosed herein.

Continuing to FIG. 2, a rear view of aspects of the exemplary embodiment of the system shown in FIG. 1 is illustrated. Note that for purposes of clarity, the first elastic portion 20 is not illustrated in FIG. 2. As further illustrated in FIG. 2, the second end 49 of the third elastic portion 40 is past the end of the arm region 16 of the detachable sleeve and generally comprises a loop that traverses the width of the back of the user's throwing hand, passing between the thumb and first finger so as to encircle the user's throwing hand.

As also further illustrated in FIG. 2, the second end 54 of the fourth elastic portion 50 may be affixed to a tab 56, and tab 56 may in turn be removably and/or adjustably affixed to the arm region 16 of the detachable sleeve 12. For example, in an embodiment the second end 54 of the fourth elastic portion 50 may be sewn or otherwise permanently affixed to tab 56. Tab 56 in turn may be configured with hook-and-loop or another fastener so as to removably affix the tab 56 to the arm region 16 adjacent or near the back of the user's throwing hand. Tab 56 may be adjusted and positioned so as to ensure the proper positioning and/or tension of the second end 54 of the fourth elastic portion 50. In this manner the tab 56 allows for an adjustable and more comfortable fit of the fourth elastic portion 50 for differing sizes of hands of various users. The tab 56 may be affixed by any desired means, such as snaps, hooks, hook-and-loop fastener, etc.

It will be understood that in some embodiments, one or more of the components discussed above for FIG. 1 and FIG. 2 may be configured differently, sized differently, and/or may not be present depending on the size, skill, strength, etc. of a user and/or the intended use to which the system 10 will be put (e.g. for prevention of injury, for rehabilitation, etc.). For example, in an embodiment, elements 50-56 discussed above may not be present depending on the size, skill, strength, etc. of a user and/or the intended use to which the system 10 will be put. Other examples are also possible and are intended to be within the scope of this disclosure.

In operation, the system 10 illustrated in FIG. 1 and FIG. 2 the user puts on, secures, and positions the detachable sleeve 12 so that the first end 22 of the first elastic portion 20 is positioned generally corresponding with the middle of the pectoralis major muscle of the user's throwing arm (in the illustrated embodiment the right arm although the disclosure herein is equally applicable to a left-handed thrower). The second end 24 of the first elastic portion 20 is positioned so as to be located generally at the bicep of the user's throwing arm. In particular, the second end 24 may in an embodiment be positioned so as to correspond with a location on the user's throwing arm that is approximately halfway down the lateral humerus and/or deltoid tuberosity.

Additionally, the first end 32 of the second elastic portion 30 is generally positioned so as to correspond with the medial border of the scapula on the user's throwing arm side (again the right side in the illustrated embodiment). In particular, in an embodiment, the first end 32 may be positioned so as to correspond with a position on the user's back that is slightly inferior and medial of superior angle of the scapula between the medial border of the scapula and the spinous process of the thoracic spine. The second end 34 of the second elastic portion 30 is positioned so as to be located generally at the bicep of the user's throwing arm. In particular, the second end 34 may in an embodiment be positioned so as to correspond with a location on the user's throwing arm that is approximately halfway down the lateral humerus and/or deltoid tuberosity.

The third elastic portion 40 is positioned with the first end 42 removably and adjustably affixed to the non-throwing side of the user's torso. The third elastic portion traverses the width of the user's back, passing under the armpit of the user's throwing arm, and extending to a second area 44 located on the arm region 16 of the detachable sleeve 12 at a position located to correspond approximately with the bicep area of the user's throwing arm. The third elastic portion 30 continues wrapping around the bicep of the user's throwing arm to the back of the arm region 16 and to a third area 46 located on the arm region 16 at approximately the tricep area of the user's throwing arm. In an embodiment, the third area 46 is located at a position corresponding to the distal humerus of the user's throwing arm. The fourth area 48 of the third elastic portion 40 runs the length of the throwing arm forearm, terminating in the illustrated embodiment as a second end 49 that forms a loop around the user's throwing hand. The second end 49 is positioned so as to traverse the width of the palm of the user's throwing hand, pass between the thumb and first finger to the back of the throwing hand, traverse the width of the back of the throwing hand and wrapping around to rejoin the fourth area 48, forming a loop around the user's throwing hand.

Note that in other embodiments (not shown) the second end 49 may circle the wrist instead of the hand. Whether the second end 49 may circle the hand or the wrist may vary depending on the size, skill, strength, etc. of a user and/or the use to which the system 10 may be put (e.g. in a game, in a practice, for rehabilitation of an injury, for injury preventions, etc.).

The fourth elastic portion 50 is positioned so that the first end 52 located at a position generally corresponding to the lateral shaft of the radius of the user's throwing arm, or about 2-6 inches from the elbow of the user's throwing arm. The middle area 53 of the fourth elastic portion 50 is generally positioned to wrap around the posterior elbow joint of the user's throwing arm and continue up along the ulna of the user's throwing arm to the second end 54. The second end 54 extends past the end of the sleeve region 16 of the detachable sleeve 12, and is positioned to pass between the thumb and first finger of the user's throwing hand to an attachment portion/tab 56 located at the back of the user's throwing hand that affixes the second end 54 to the back side of the arm region 16 of the detachable sleeve.

When the user then engages in a throwing motion, such as pitching a baseball, throwing a football, etc., the wind-up portion of the throwing motion generally includes stepping forward and/or rotating the torso to position the throwing arm away from the target. During this wind-up motion, the humerus bone in the throwing arm is horizontally abducted and externally rotated, causing tension to be placed on the first elastic portion 20, third elastic portion 40 and fourth elastic portion 50 in varying order and to varying degree depending on the user's particular throwing motion, range of motion, amount of wrist cock, etc. In an embodiment, this wind-up motion will cause tension to load first in the first elastic portion 20, then the third elastic portion 40, and then the fourth elastic portion 50. This tension helps prevent an overextension of the UCL during the wind-up, during a warm-up, during throwing practice, and/or during rehabilitation of an injury to the UCL.

When the user then engages in the throwing motion, the tensions in the first elastic portion 20, third elastic portion 40 and fourth elastic portion 50 are unloaded (e.g. by releasing the potential energy from the loaded elastic material), assisting the varying muscles in the arm and shoulder that provide stability to UCL during the forward motion of the arm, during the rotation of the lower arm around the axis of the upper arm, and during the pronation of the hand leading to the release of the ball (or other object thrown). Again, the order, degree, and speed of the unloading may vary depending on the particular user and the throwing motion of the user.

In an embodiment, the unloading of the first elastic portion 20 first assists in the process of the throwing arm moving forward with horizontal adduction, then the third elastic portion 40 unloads to assist in internally rotating the humerus which also reinforces the UCL. Finally, the fourth elastic portion 50 unloads, assisting the forearm flexors from the medial epicondyle of the humerus up to the hand in the release of the object being thrown, increasing the throwing velocity. In other embodiments, the first elastic portion 20, third elastic portion 40 and fourth elastic portion 50 may unload in slightly differing orders and/or one or more may unload at substantially the same time. These unloading tensions provide external assistance to the arm and shoulder muscles supporting the elbow and allow the user to engage in a full throwing motion while at the same time lessening the strain on the UCL throughout the entirety of the motion. This further assists in preventing injuries either acute such as during warm-up, or from repetitive motions and/or overuse.

Similarly, during the follow-through of the throwing motion, one or more of the elastic portions 20, 30, 40 may re-load with tension as the user's arm straightens, again in varying order and to varying degrees depending on the user's particular throwing motion, range of motion, etc. For example, the second elastic portion 30 and the third elastic portion 40 may begin reloading tension during the horizontal adduction and internal rotation of the arm during the follow through. This re-loading of tensions helps prevent overextensions of the UCL or the elbow, and provides additional assistance to the arm and shoulder muscles supporting the UCL.

For example in children with developing muscles and ligaments, the loading and unloading of the elastic portions 20, 30, 40, 50 of the system 10 during wind-up, throwing motion, and/or follow-through allow for full ranges of motion and full strength throwing while reducing the strains that lead to overuse injuries such as little league elbow. Similarly, for individuals with injured UCLs, these loading and unloading of the elastic portions 20, 30, 40, 50 in the system 10 during the throwing motion allow for rehabilitation treatments to include a full throwing motion and/or re-strengthen the muscles of the throwing motion while preventing re-injury or further injury to the UCL. A regimen of treatment may call for stronger elastic portions 20, 30, 40, 50 at the beginning of treatment with lessening strengths of the elastic portions 20, 30, 40, 50 as treatment progresses.

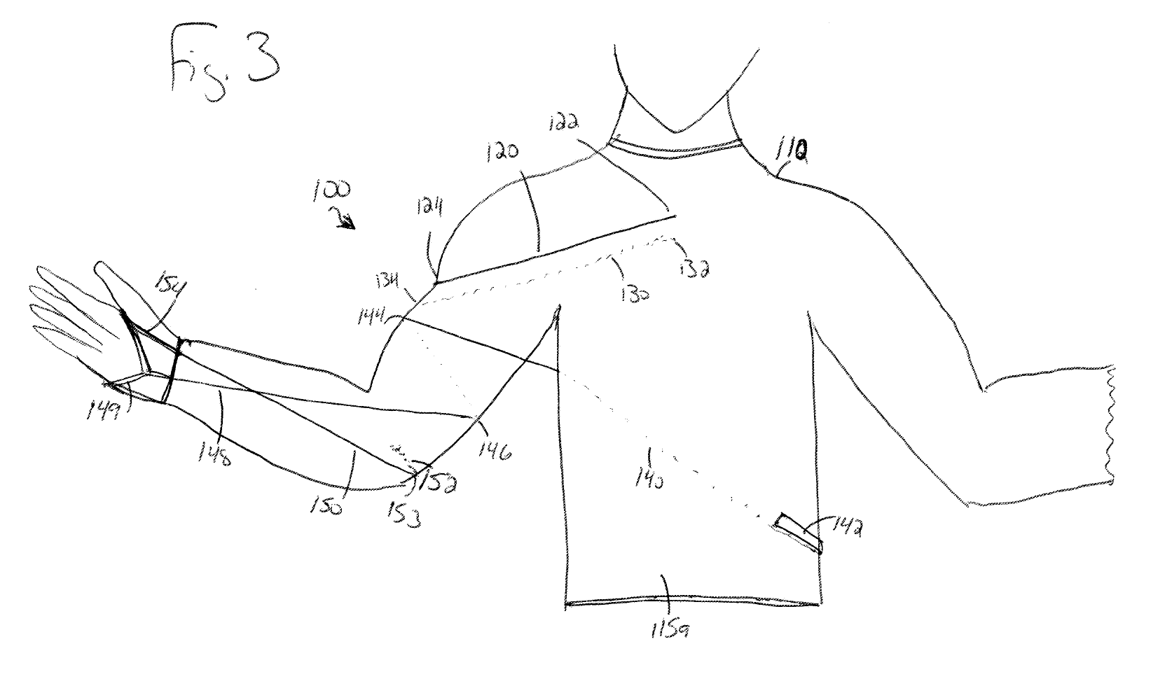

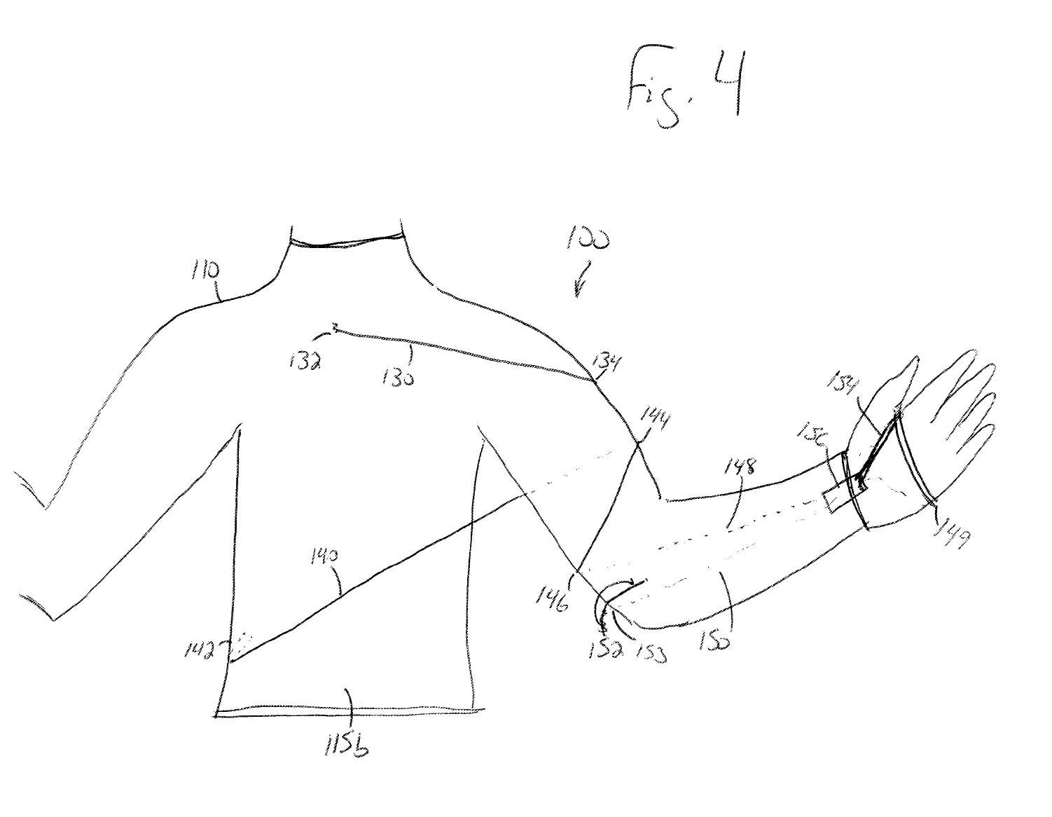

FIGS. 3 and 4 illustrate a front view and rear view, respectively, of another exemplary embodiment of a system 100 that provides external assistance for a throwing motion. In the embodiment illustrate in FIGS. 3 and 4, a first elastic portion 120, second elastic portion 130, third elastic portion 140, and fourth elastic portion 150 are incorporated into a shirt 110 with a front side 115a and a rear side 115b rather than the detachable sleeve 12 illustrated in FIGS. 1-2 discussed above. However, the principles and configuration of the first elastic portion 120, second elastic portion 130, third elastic portion 140, and fourth elastic portion 150 are similar to those discussed above for the embodiment of FIGS. 1-2.

In the embodiment of FIGS. 3 and 4, the shirt 110 may be comprised of any suitable material, or combination of materials that provide the support, fit, and positioning of the elastic portions 120, 130, 140, 150 as discussed above. For example in an embodiment the shirt 110 may be a compression shirt configured to be form fitting to a user's body and throwing arm. Various portions of shirt 110 may be made of differing materials, differing densities of material, differing compression levels of materials, differing thicknesses of materials, etc. as desired. Further, one or more portions of shirt 110 may include cut-outs, slits, vents, mesh material, etc., to allow for air and/or moisture ventilation.

Additionally, as illustrated in FIGS. 3 and 4, one or more of the elastic portions 120, 130, 140, 150 may be affixed to the shirt 110, by creating a channel or holder to hold the elastic portions 120, 130, 150 in the proper positions, while also allowing the elastic portions 120, 130, 150 to properly load and unload as discussed above. For example, in some embodiments the elastic portions 120, 130, 150 may be implemented as elastic cords and/or bands of similar or dissimilar material, elasticity, thickness, etc. placed between layers of fabric for the shirt 110 and held into place with stitching on either side of the elastic portions 120, 130, 150 to keep them properly positioned.

Note that in some embodiments, the shirt 110 may instead implement external elastic portions 120, 130, 150 similar to the first elastic portion 20, second elastic portion 30, and fourth elastic portion 50 discussed above for FIGS. 1 and 2. Similarly, the elastic cords and/or bands discussed for FIGS. 3 and 4 could also be implemented with the detachable sleeve 12 of FIGS. 1 and 2 if desired.

As also discussed above with respect to FIGS. 1 and 2, the second end 154 of the fourth elastic portion 150 may removably attach at the back of the user's hand to a tab 156 that is connected to the shirt 110 such as at a cuff or end of a sleeve of the shirt 110, allowing for an adjustable fit of the fourth elastic portion 150. Additionally, the second end 149 of the third elastic portion 140 may extend from the end of a sleeve of the shirt 110 to encircle the throwing hand of the user as discussed above with respect to the second end 49 of FIGS. 1-2. The system 100 of FIGS. 3 and 4 operates similarly to the system 10 discussed above, providing the same benefits and assistance to the arm and shoulder muscles that support the UCL during the entire throwing motion.

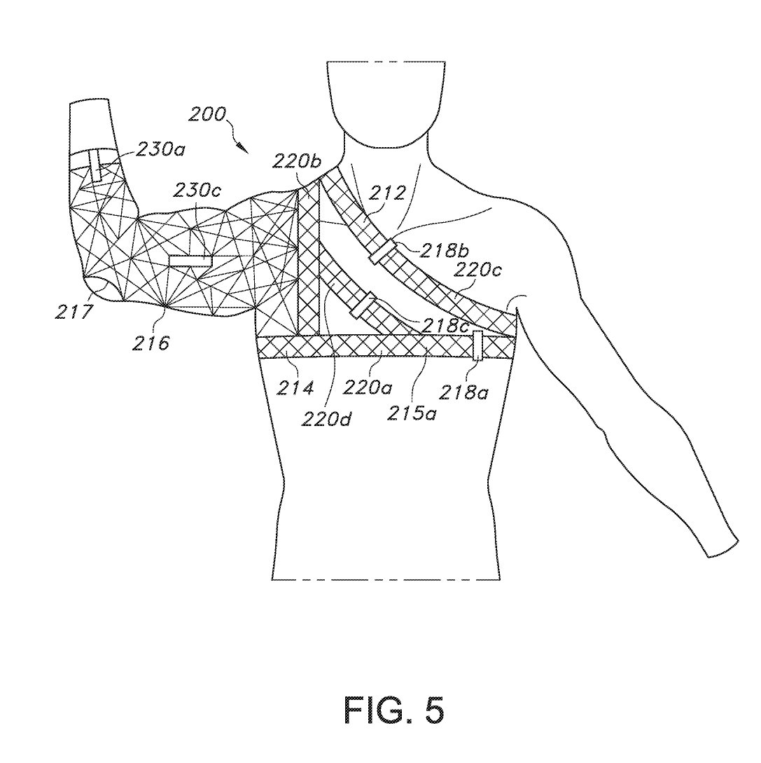

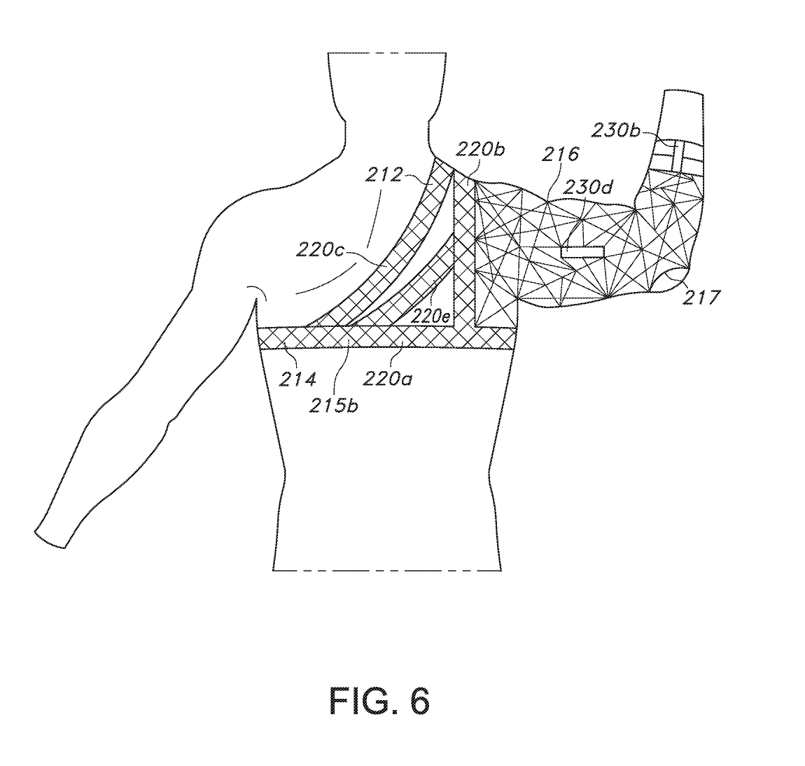

FIGS. 5 and 6 illustrate a front and rear view, respectively, of yet another exemplary embodiment of a system 200 that provides external assistance for a throwing motion. In the embodiment illustrated in FIGS. 5 and 6, the detachable sleeve 212 is comprised of an elastomer (e.g., a silicone resin) mesh arm region 216 that extends from a torso region 214. In this exemplary embodiment, the elastomer mesh of the arm region 216 performs the same or similar function(s) as those attributed to the first elastic portion 20, the second elastic portion 30, the third elastic portion 40, and/or the fourth elastic portion 50 of the system 10 described above.

When the user engages in a throwing motion, such as pitching a baseball, throwing a football, etc., the wind-up portion of the throwing motion generally includes stepping forward and/or rotating the torso to position the throwing arm away from the target. During this wind-up motion, the humerus bone in the throwing arm is horizontally abducted and externally rotated, causing tension to be placed on the elastomer mesh of the arm region 216 to a varying degree depending on the user's particular throwing motion, range of motion, amount of wrist cock, etc. In this embodiment, the wind-up motion will cause tension to load in the elastomer mesh of the arm region 216. This tension helps prevent an overextension of the UCL and/or rotator cuff during the wind-up, during a warm-up, during throwing practice, and/or during rehabilitation of an injury to the UCL and/or rotator cuff.

When the user engages in a throwing motion, the elastomer mesh arm region 216 of the detachable sleeve 212 can assist the varying muscles (e.g., the rotator cuff) in the arm and shoulder that provide stability to the UCL, elbow joint, and/or glenohumeral joint during the forward motion of the arm, during the rotation of the lower arm around the axis of the upper arm, and during the pronation of the hand leading to the release of the ball (or other object thrown). In some implementations, the elastomer mesh arm region 216 of the detachable sleeve may assist the various muscles in the arm and shoulder as described above during other similar arm motions. Other similar arms motions may include a swinging motion, such as tennis, golf, handball, volleyball, etc. that generally involve a winding-up motion, an acceleration of the arm, hitting the ball, and a follow-through.

As shown in FIGS. 5 and 6, the system 200 includes a detachable sleeve 212 configured to be worn on the throwing arm of a user. In the embodiment illustrated in FIG. 5, the detachable sleeve 212 is configured and adapted to be worn on the right arm; however in the event that the user throws left handed, the detachable sleeve 212 may be configured and adapted to be worn on the left arm if desired as would be understood by one of ordinary skill in the art.

The detachable sleeve 212 shown in FIG. 5 includes a torso region 214 configured to be attached to the torso of the user and an elastomer mesh arm region 216 configured to cover at least a portion of the throwing arm of the user when the detachable sleeve 212 is in use.

In the embodiment illustrated in FIG. 5, the arm region 216 comprises a long sleeve of elastomer mesh that covers the user's throwing arm to the middle of the forearm. However, in other embodiments, the arm region 216 may be longer and extend to the user's wrist, or shorter. In some embodiments, the elbow portion of the arm region 216 may include an opening 217 therethrough large enough for the elbow of a user to extend through (see, e.g., FIG. 5). In this way, the elastomer mesh of the arm region 216 will not interfere with the rotation of the elbow during a throwing motion.

As shown in FIG. 5, the elastomer mesh of the arm region 216 comprises a plurality of openings that extend therethrough. In some embodiments, the shapes of the openings in the elastomer mesh are selected to optimize (e.g., increase or decrease based on use) the buildup of potential energy that can be converted to kinetic energy during a throwing motion. In this way, the amount of kinetic energy generated to assist the user during a throwing motion may be varied. In some embodiments, the elastomer mesh of the arm region 216 may comprise a plurality of geometric shaped openings, for example triangles (see, e.g., FIG. 5). Triangle shaped openings may be used to optimize stretch lines and thereby the buildup of potential energy that can be converted to kinetic energy during a throwing motion while also minimizing the amount of material used to construct the arm region 216. In some embodiments, the elastomer mesh of the arm region 216 may comprise a plurality of geometric shaped openings and/or non-geometric shaped openings. Additionally, the openings in the elastomer mesh of the arm region 216 allow for moisture and/or air ventilation.

In some embodiments, the arm region 216 may be made of an elastomer material other than a silicone resin (e.g., a silicone rubber and/or other like materials). The tensile strength of the elastomer (e.g., a silicone resign) used to form the arm region 216 affects (e.g., increases or decreases) the speed of a user's throw. Note that in varying embodiments, one or more portions of the arm region 216 may be made of the same or different materials, may be the same or different thickness or width, may have the same or different amounts (or moduli) of elasticity, etc. as desired and in accordance with the systems and methods disclosed herein.

In some implementations, the configuration of the elastomer mesh of the arm region 216 and/or the strength of the elastomer selected may be used to limit the user's range of motion. In this way, the detachable sleeve 212 may be used as a safety device. In some implementations, the configuration of the elastomer mesh of the arm region 216 and/or the strength of the elastomer may not limit the user's range of motion.

As shown in FIGS. 5 and 6, the arm region 216 may include a plurality of fasteners 230 thereon. In some embodiments, there may be a first fastener 230a and a second fastener 230b positioned opposite each other, adjacent the distal end of the arm region 216 (see, e.g., FIGS. 5 and 6). The fasteners 230a, 230b allow the distal portion of the arm region 216 to be adjustably secured to fit on forearm of varying sizes, diameters, etc . . . and/or allow the arm region 216 to be positioned properly on a user.

As shown in FIGS. 5 and 6, in some embodiments, there may be a third fastener 230c and a fourth fastener 230d positioned opposite each other at a location that corresponds to the bicep region of the user's throwing arm. The fasteners 230c, 230d allow the portion of the arm region 216 positioned about the bicep to be adjustably secured to fit on bicep of varying sizes, diameters, etc... and/or allow the arm region 216 to be positioned properly on a user.

The fasteners 230 (i.e., 230a, 230b, 230c, 230d ) may be any appropriate mechanism for, or method of, securely attaching the arm region 216 about the throwing arm of the user. Examples of fasteners 230 include snaps, hooks, hook-and-loop fasteners, buttons, etc. capable of adjustably securing the arm region 216 about the arm of the user.

As shown in FIGS. 5 and 6, the torso region 214 of the detachable sleeve 212 includes a front side 215a and a back side 215b configured to secure about the torso of a user. In some embodiments, the torso region 214 may be comprised of a horizontal strap 220a, a vertical strap 220b, a first angle strap 220c, a second angled strap 220d, and a third angled strap 220e (collectively referred to as straps 220) (see, e.g., FIGS. 5 and 6). In some embodiments, the straps 220 may be configured to adjustably position and/or secure the torso region 214 of the detachable sleeve 212 about the torso of a user. In this way, the user may adjust the fit of the torso region 214 on his or her body during use. In some embodiments, the torso region 214 of the detachable sleeve 212 may be constructed the same as, or similar to, the torso region 14, 214 of the detachable sleeve 12, 112 previously described herein.

In some embodiments, the horizontal strap 220a is configured to wrap around the torso of a user (see, e.g., FIGS. 5 and 6).

In some embodiments, the vertical strap 220b extends from a first position on the horizontal strap 220a located on a front side 215a of the torso region 214, over the shoulder of the user, to a second position on the horizontal strap 220a located on the back side 215b of the torso region 214 (see, e.g., FIGS. 5 and 6). In this way, the vertical strap 220b supports the horizontal strap 220a of the torso region 214. The elastomer mesh arm region 216 of the detachable sleeve 212 is connected to and extends from the vertical strap 220b and a portion of the horizontal strap 220a, as shown in FIGS. 5 and 6.

In some embodiments, the first angled strap 220c extends from a third position on the horizontal strap 220a located on a front side 215a of the torso region 214, over the shoulder of the user, to a fourth position on the horizontal strap 220a located on the back side 215b of the torso region 214 (see, e.g., FIGS. 5 and 6).

In some embodiments, the second angled strap 220d extends from a fifth position on the horizontal strap 220a located on a front side 215a of the torso region 214 to a first position on the vertical strap 220b located on a front side 215a of the torso region 214.

In some embodiments, the third angled strap 220e extends from a sixth position on the horizontal strap 220a located on a back side 215b of the torso region 214 to a second position on the vertical strap 220b located on a back side 215b of the torso region 214.

In some embodiments, the straps 220 may be made of any appropriate material, such as nylon that will allow for a snug fit to a user when the detachable sleeve 212 is worn. Accordingly, the material of the detachable sleeve 212 may be relatively inelastic as desired to allow for a snug fit. Additionally, in some embodiments the torso region 214 of the detachable sleeve 212 may comprise a different material, different thickness of material, different density of material, different elasticity, etc. than the arm region 216 of the detachable sleeve 212.

A shown in FIG. 5, in some embodiments, the horizontal strap 220a, the first angled strap 220c, and the second angled strap 220d may each include a connector 218a, 218b, 218c (collectively connectors 218), respectively, thereon configured to adjust the length thereof. In this way, a user may adjust the fit of the torso 214 region when the detachable sleeve 212 is being worn. Each of the connectors 218 may be any appropriate mechanism for, or method of, adjusting the length of a strap 220 and/or connecting two portions of a strap 220 together when the user places his or her arm into the detachable sleeve 212. Example connectors 218 include buckles, triglide rings, snaps, hooks, hook-and-loop fasteners, buttons, etc. capable of adjustably connecting two portions of a strap 220 together. In the illustrated embodiment of FIG. 5, each connector 218 is located on the front side 215a of the torso region 214 so that they may be easily adjusted when the detachable sleeve 212 is being secured to the user. In other embodiments, the connectors 218 may be located on the back side 215b, on the side of the user's torso, or anywhere that allows for the detachable sleeve 212 to be secured to the user.

In some implementations, the detachable sleeve 212 may be configured so that one could be worn on each arm of the user. In some implementations, the torso region of a detachable sleeve may include two elastomer mesh arm regions 216 thereon configured to cover at least a portion of each throwing arm of the user when the detachable sleeve is being worn.

Thus, by way of a non-limiting example, in one embodiment, the arm region 216 may comprise a first material (e.g., silicone resign), a first density of material and/or a first thickness of material, while the torso region 214 may comprise a second material (e.g., nylon straps), a second density of material and/or a second thickness of material as desired.

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown, or in sequential order, or that all illustrated operations be performed, to achieve desirable results.

1. A system for providing external assistance to arm and shoulder muscles, comprising:
a detachable sleeve comprising a torso region configured to receive at least part of a torso of a user and an arm region configured to receive at least part of an arm of the user;
wherein the arm region comprises an elastomer mesh that extends from the torso region of the detachable sleeve.

2. The system of claim 1, wherein the elastomer mesh of the arm region is a silicone resin.

3. The system of claim 1, wherein the elastomer mesh of the arm region comprises a plurality of geometric shaped openings.

4. The system of claim 3, wherein the elastomer mesh of the arm region comprises a plurality of triangle shape openings.

5. The system of claim 1, further comprising an opening in an elbow portion of the arm region of the detachable sleeve configured to allow the elbow of a user to extend therethrough.

6. The system of claim 1, wherein the arm region of the detachable sleeve further comprises at least a first fastener and a second fastener positioned thereon at a location corresponding to the bicep region of the arm of the user, the first fastener and the second fastener are configured to adjustably secure at least a portion of the arm region of the detachable sleeve about the bicep of the user.

7. The system of claim 6, wherein the arm region of the detachable sleeve further comprises at least a third fastener and a fourth fastener positioned adjacent a distal end thereof, the third fastener and the fourth fastener are configured to adjustably secure the distal end of the arm region of the detachable sleeve about the forearm of a user.

8. The system of claim 1, wherein the torso region of the detachable sleeve comprises a plurality of nylon straps.

9. The system of claim 1, wherein the detachable sleeve further comprises a second arm region configured to receive at least part of an arm of the user; wherein the second arm region comprises an elastomer mesh that extends from the torso region of the detachable sleeve.
